(19) United States
(12) Patent Application Publication
(10) Pub. No.: US 2015/0141945 A1
(43) Pub. Date: May 21, 2015

(54) Disposable Feminine Hygiene Utility

(71) Applicant: Ravit Emergi, Raanana (IL)

(72) Inventor: Ravit Emergi, Raanana (IL)

(21) Appl. No.: 14/085,907

(22) Filed: Nov. 21, 2013

(51) Int. Cl. A61F 13/472

(52) U.S. Cl. CPC A61F 13/47218
USPC 604/369; 604/385.01

(57) ABSTRACT

A disposable feminine hygiene utility (10), comprising: a sanitary napkin (12) to be placed between an undergarment (26) of a user and a user's body; a permeable longitudinal sleeve (14), connected to the sanitary napkin (12); an absorbent pillow (20), enclosed by the permeable longitudinal sleeve (14); and first and second wires (18), each connected to another end of the absorbent pillow (20), for adjusting a position of the absorbent pillow (20) in relation to the longitudinal sleeve (14), with reference to a feminine anatomic section.

### FIELD OF THE INVENTION

The present invention relates to the field of feminine hygiene. More particularly, the invention relates to a disposable feminine hygiene utility.

### BACKGROUND OF THE INVENTION

“Feminine hygiene” is an umbrella term used to describe toilet products for menstrual fluids collection.

Regular menstruation (also called eumenorrhea) lasts for a few days, usually three to five days, but anywhere from two to eight days is considered normal. The average menstrual cycle is 28 days long from the first day of one menstrual period to the first day of the next. A normal menstrual cycle is typically 21 to 35 days between menstrual periods.

The average volume of menstrual fluid during a monthly menstrual period is 35 milliliters (or four to six tablespoons of menstrual fluid) with 10-80 milliliters considered typical. Menstrual fluid contains blood, cervical mucus, vaginal secretions, and endometrial tissue. The color of menstrual fluid is reddish-brown, a slightly darker color than blood.

Most females use something to absorb or catch their menses. There are a number of different disposable menstrual absorption products available for use today. Sanitary napkins and tampons are the most common disposable absorption products in use.

Sanitary napkins, or pads, are somewhat rectangular- shaped pieces of material worn in the underwear to absorb menstrual flow, often with “wings”—pieces that fold around the undergarment, or an adhesive backing to hold the pad in place. Disposable sanitary napkins may contain wood pulp or gel products, usually bleached and with a plastic lining. Most sanitary napkins are held in place by adhesive or wings.

A menstrual pad or a sanitary napkin is worn during a woman's period to absorb her menstrual flow. It is worn externally, between the vulva and a woman's undergarment. Menstrual pads are made from a range of different materials.

Tampons, such as disclosed by U.S. Pat. No. 6,890,325 and U.S. Pat. No. 6,316,688, are disposable cylinders of treated rayon or cotton blends or all-cotton fleece, usually bleached, that are inserted into the vagina, to absorb menstrual flow. A regular tampon is usually built out of a mass of cotton, rayon, or a mixture of the two.

Tampons come in various sizes, which are related to their absorbency ratings and packaging. The outward appearance of a tampon is similar in all brands, but their absorbency varies. The two main differences are in the way the tampon expands when in use: some types of tampons will expand axially (increase in length), while others will expand radially (increase in diameter). All tampons have a thread for removal and some have an additional outer cover to aid insertion and withdrawal. Tampons are sold individually wrapped to keep them sanitary. Tampons can range in size from 1½ to 4½ inches.

Tampons have various associated health risks. Tampons were found to be a cause of toxic shock syndrome (TSS) cases. The blame is in their high absorbency, as well as in their overnight use, for increased incidences of toxic shock syndrome. Materials used in most modern tampons are so highly absorbent that they pose the risk of absorbing the natural discharge and upsetting the natural moisture balance, which is what enables toxic shock syndrome to occur.

Some women experience a heavier-than-average menstrual flow. Typical to this group is a more observable shedding of the uterus's endometrium lining during menstruation. The shed endometrium lining appears here as small pieces of tissue mixed with the blood. These pieces of tissue are in fact common, but occur more frequently in women who experience a heavier-than-average menstrual flow.

The health risks of the use of tampons and the disadvantages of sanitary napkins with women who experienced leakage, entails the need of a better solution to the problem of absorbing menstrual fluids.

All the methods described above have not yet provided satisfactory solutions to the problems of disposable feminine hygiene utilities.

It is an object of the present invention to provide a solution to the above-mentioned and other problems of the prior art.

Other objects and advantages of the invention will become apparent as the description proceeds.

### SUMMARY OF THE INVENTION

In one aspect, the present invention is directed to a disposable feminine hygiene utility (10), comprising:
a sanitary napkin (12) to be placed between an undergarment (26) of a user and a user's body;
a permeable longitudinal sleeve (14), connected to the sanitary napkin (12);
an absorbent pillow (20), enclosed by the permeable longitudinal sleeve (14); and
first and second wires (18), each connected to another end of the absorbent pillow (20), for adjusting a position of the absorbent pillow (20) in relation to the longitudinal sleeve (14), with reference to a feminine anatomic section.

The adjusting may be carried out without contact between the absorbent pillow (20) and a user's body, resulting in preventing irritation of the user's body.

Preferably, the sleeve (14) is structured in the sanitary napkin (12).

The sleeve (14) may be attached to the sanitary napkin (12) by sewing, welding, adhering, and so on.

Preferably, the absorbent pillow (20) is flattened at the bottom thereof

According to one embodiment of the invention, the absorbent pillow (20) is made of spongy material, thereby allowing the pillow to deform according to a user's body that touches the absorbent pillow (20).

The utility may be further disposed in a pack (16), thereby keeping the utility sterilized until use.

Preferably, the pack (16) is designed to allow placing therein a used utility, and keeping thereof sealed.

In order to prevent smooth adjusting of the location of the absorbent pillow, the side of the pillow that turns to the undergarment may comprise a waxed layer. This provides also an additional layer that isolates the undergarment from menstrual fluids and the like.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

### BRIEF DESCRIPTION OF THE DRAWINGS

Embodiments and features of the present invention are described herein in conjunction with the following drawings:

FIG. 1 schematically illustrates a FHU 10, according to one embodiment of the invention.

FIG. 2 schematically illustrates a FHU 10′, according to a further embodiment of the invention.

FIG. 3 schematically illustrates the disposable feminine hygiene utility of FIG. 2, in which the major parts thereof are separated.

FIG. 4 schematically illustrates the result of cross-section A-A of FIG. 2.

FIG. 5 schematically illustrates a FHU 10″, according to another embodiment of the invention.

FIG. 6 schematically illustrates the FHU 10″ of FIG. 5, attached to undergarment 26. FIG. 6 also defines a cross-section B-B.

FIG. 7 schematically illustrates the result of cross-section B-B of FIG. 6.

FIG. 8 schematically illustrates a package FHU 10″, according to another embodiment of the present invention.

It should be understood that the drawings are not necessarily drawn to scale.

### DETAILED DESCRIPTION OF PREFERRED EMBODIMENTS

The present invention will be understood from the following detailed description of preferred embodiments, which are meant to be descriptive and not limiting. For the sake of brevity, some well-known features, methods, systems, procedures, components, circuits, and so on, are not described in detail.

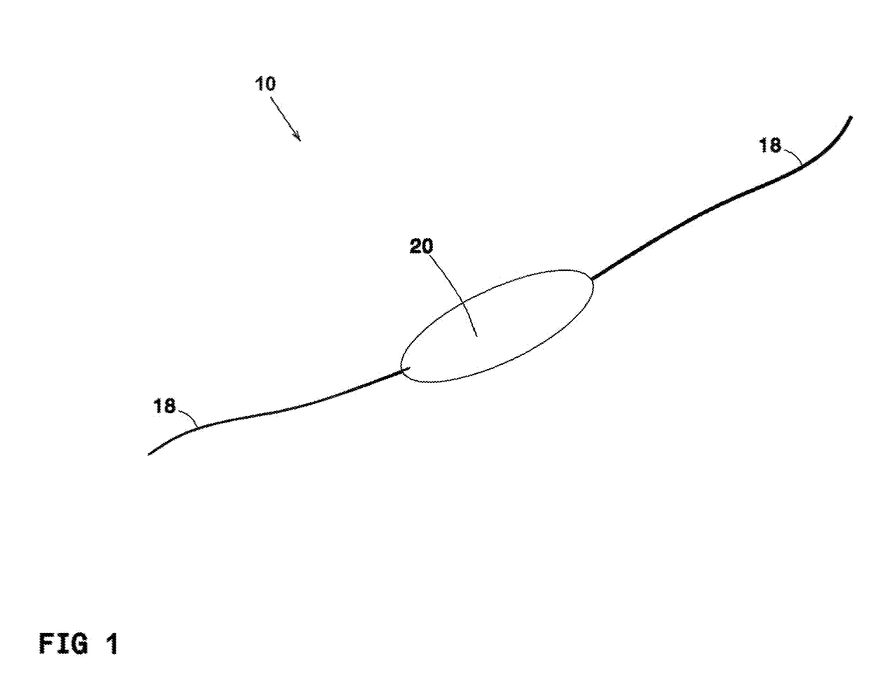

FIG. 1 schematically illustrates a FHU 10, according to one embodiment of the invention.

The acronym FHU refers herein to a Disposable Feminine Hygiene Utility.

The FHU 10 of FIG. 1 comprises an elongated absorbent pillow, which is marked herein by reference numeral 20. On each end of the absorbent pillow 20 is connected a wire, which is marked herein by reference numeral 18.

In order to use the FHU 10, the user places the FHU 10 between an undergarment of the user and hers body.

The position of the absorbent pillow with reference to a feminine anatomic section can be changed after placing FHU 10 between an undergarment of the user and the user's feminine anatomic section. Thus, if after a while the user feels uncomfortable with the location of the pillow, the location can be changed in a desired direction by pulling the corresponding wire 18.

In order to prevent smooth adjusting of the location of the absorbent pillow, the side of the pillow that turns to the undergarment may comprise a waxed layer. This provides also an additional layer that isolates the undergarment from menstrual fluids and the like.

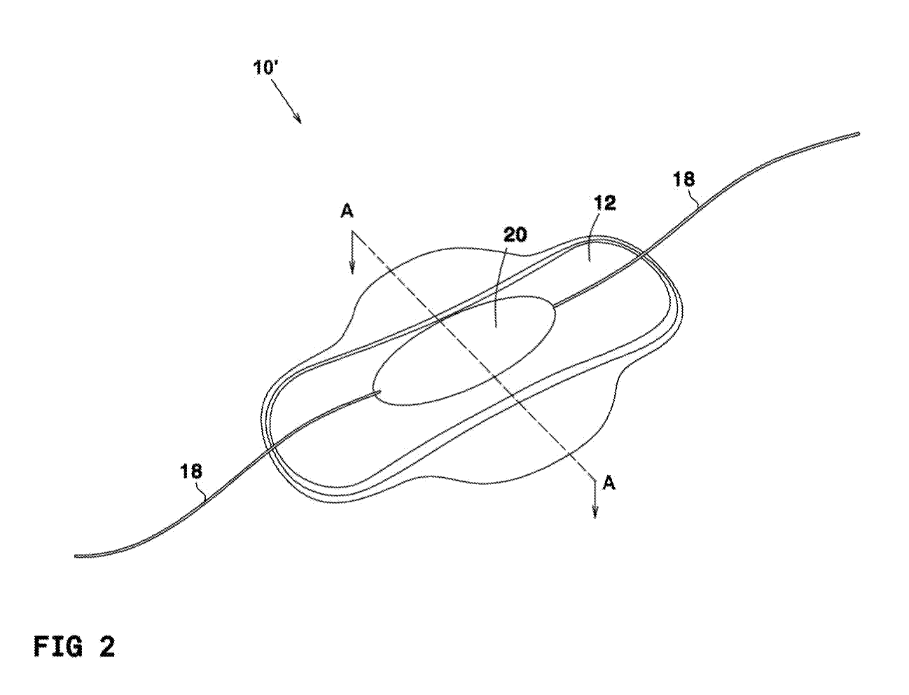

FIG. 2 schematically illustrates a FHU 10′, according to a further embodiment of the invention.

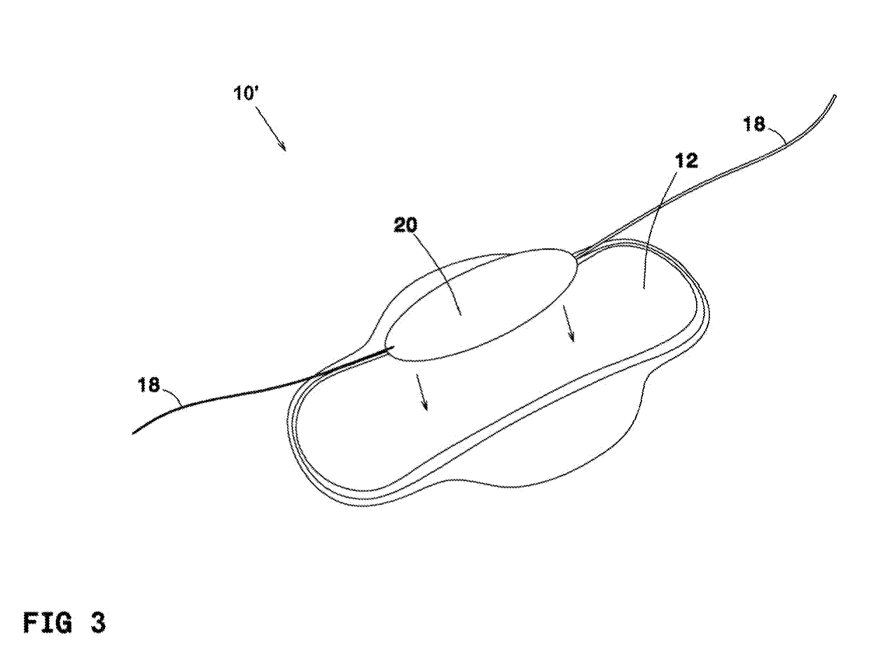

FIG. 3 schematically illustrates the disposable feminine hygiene utility of FIG. 2, in which the major parts thereof are separated.

FHU 10′ comprises two major parts: a sanitary napkin, which is marked herein by reference numeral 12, and an elongated absorbent pillow, marked herein by reference numeral 20 (which comprises two wires 18, as in the embodiment of FIG. 1).

The absorbent pillow 20 is a separate entity from the sanitary napkin 12, as illustrated in FIG. 2. In order to use the FHU 10′, absorbent pillow 20 is placed in about the center of the sanitary napkin 12, such that the longitudinal orientation thereof is parallel to the longitudinal orientation of the sanitary napkin 12.

After the FHU 10′ is attached to the undergarment of a user, and the user wears the undergarment, wires 18 are useful for shifting the location of the absorbent pillow 20 with reference to the sanitary napkin 12, in order to place the absorbent pillow 20 in a desired location.

As the sanitary napkin 12 comprises an absorption pad, the absorbent pillow 20 increases the absorption ability of the FHU 10′. Furthermore, a user thereof may dispose of the absorbed pillow 20, and still use the sanitary napkin 12, which is less absorbed. Thus, instead of replacing the entire FHU 10′, the user may dispose of the absorbent pillow, which is faster than replacing the entire FHU 10′.

The arrows in FIG. 3 illustrate how absorbent pillow 20 is placed on sanitary napkin 12.

FIG. 2 also defines a cross-section A-A.

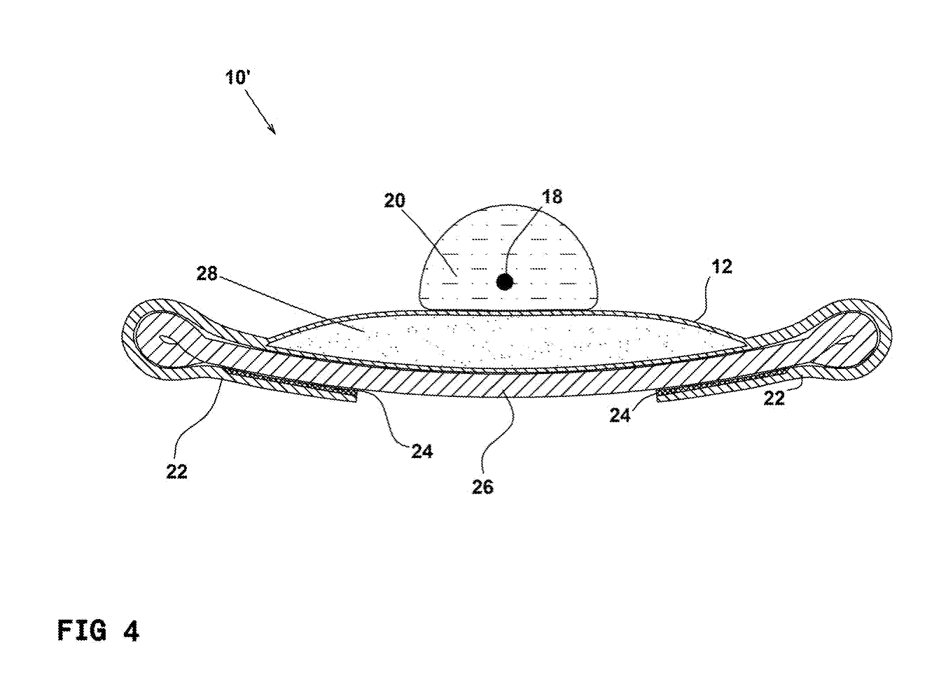

FIG. 4 schematically illustrates the result of cross-section A-A of FIG. 2.

As illustrated, the bottom of the absorbent pillow 20 is substantially flat, in order to correspond to the flat form of the absorption pad 28 of the sanitary napkin. The flat bottom form of the absorbent pillow may be achieved by a design which provides the flatness. Alternatively, the absorbent pillow may be made of spongy material and the like, which allows deforming the absorbent pillow according to the pressures thereon.

The sanitary napkin 12 is attached to undergarment 26 by wings 22, which are binding undergarment 26 from both of its sides, thereby providing additional protection against leakage of menstrual fluid and help to secure sanitary napkin 12 in place. Adherence material 30 is used here to fix wings 22 of sanitary napkin 12 onto undergarment 26, while wings 22 are wrapped around the sides of undergarment 26 to add additional leak protection and help secure sanitary napkin 12 in place.

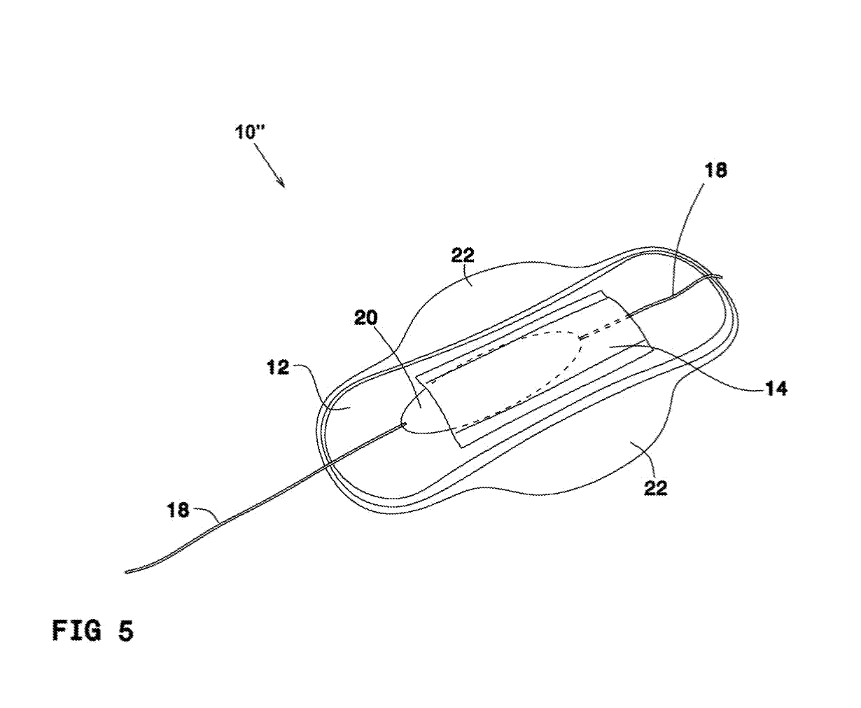

FIG. 5 schematically illustrates a FHU 10″, according to another embodiment of the invention.

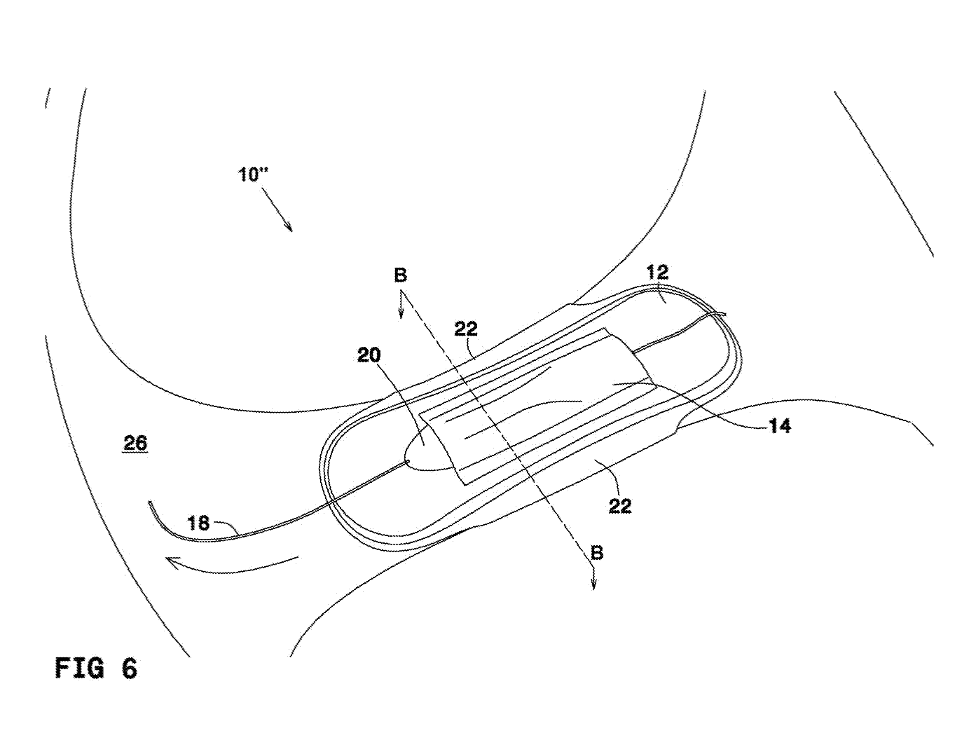

FIG. 6 schematically illustrates the FHU 10″ of FIG. 5, attached to undergarment 26. FIG. 6 also defines a cross-section B-B.

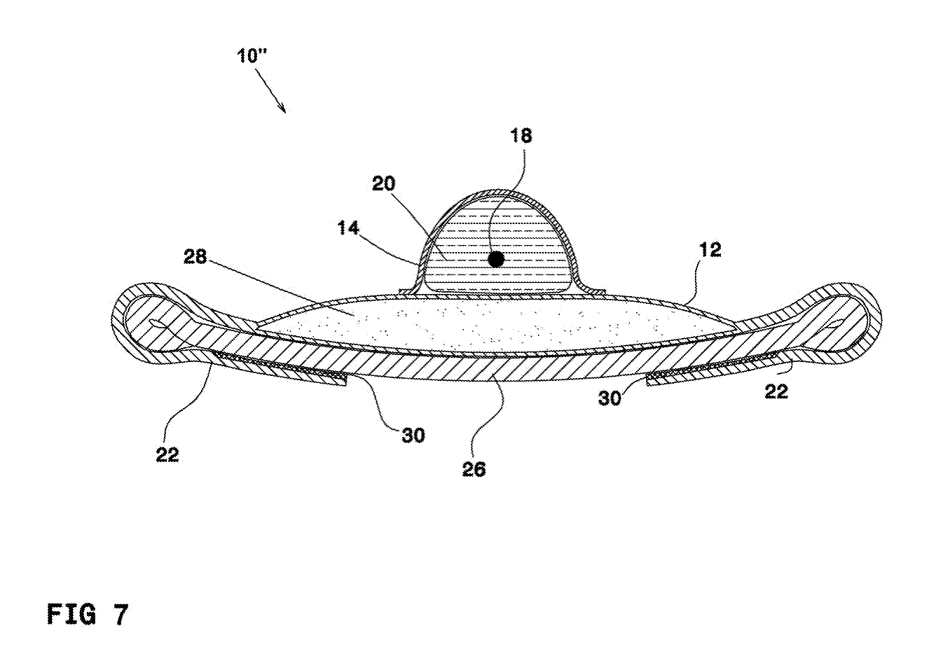

FIG. 7 schematically illustrates the result of cross-section B-B of FIG. 6.

The FHU, which is referred to herein by reference numeral 10′, comprises a structured sleeve 14, to attain the centered position of absorbent pillow 20 with regard to the sanitary napkin 12.

As sleeve 14 separates between a user's body and the absorbent pillow 20, the operation of shifting the pillow is carried out without a contact between the pillow and the user's body, i.e., without irritating the user's body.

Feminine hygiene utility with a sleeve provides some different characteristics derived from its structural construction. The sleeve 14 is made from permeable material to allow leaking fluid to pass through.

According to one embodiment of the invention, the sleeve (14) is structured in the sanitary napkin (12).

The structure can be carried out by adhering the sleeve to the napkin, sewing the sleeve to the napkin, welding the sleeve to the napkin, and so on.

The arrow in FIG. 6 illustrates the direction of pulling absorbent pillow 20 by a user, in order to adjust its location with reference to the sanitary napkin 12 and the user's body.

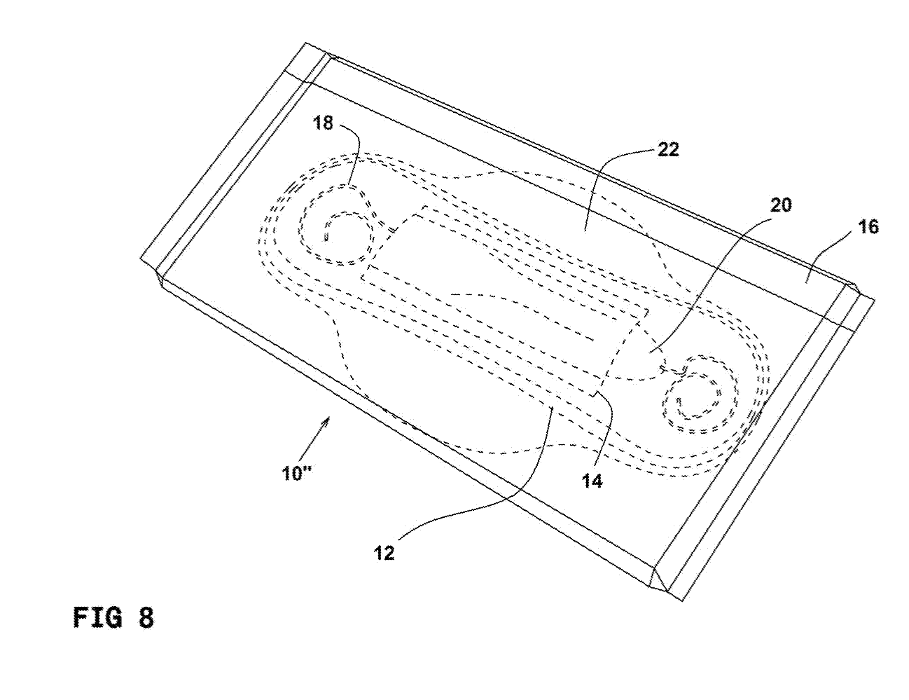

FIG. 8 schematically illustrates a package FHU 10″, according to another embodiment of the present invention.

FHU 10″ is individually wrapped, and stored in a pack 16. The wires 18 are rolled such that they do not pass the borders of the sanitary napkin 12. The pack may be made of cellophane, nylon, and so on. The pack may also be made of cardboard or plastic, in a box form.

According to one embodiment of the invention, the pack (16) is designed for storing a plurality of FHUs.

According to one embodiment of the invention, the pack is designed to enable sealing thereof after opening. This way, a user may place a used utility in the pack thereof, thereby preventing contamination from the used FHU.

In the figures and/or description herein, the following reference numerals have been mentioned:

numeral 10 denotes a disposable feminine hygiene utility (FHU), according to one embodiment of the invention;

numeral 10′ denotes a disposable feminine hygiene utility (FHU), according to a further embodiment of the invention;

numeral 10″ denotes a disposable feminine hygiene utility (FHU), according to a further embodiment of the invention;

numeral 12 denotes a sanitary napkin;

numeral 14 denotes a permeable sleeve, preferably structured;

numeral 16 denotes a pack of a FHU;

numeral 18 denotes a wire connected to one end of absorbent pillow 20;

numeral 20 denotes an absorption pillow;

numeral 22 denotes “wings” of a sanitary napkin;

numeral 24 denotes adherence material, for adhering sanitary napkin 12 to undergarment 26;

numeral 26 denotes an undergarment;

numeral 28 denotes an absorbent pad of sanitary napkin 12; and

numeral 30 denotes adherent material.

The foregoing description and illustrations of the embodiments of the invention has been presented for the purposes of illustration. It is not intended to be exhaustive or to limit the invention to the above description in any form.

Any term that has been defined above and used in the claims, should to be interpreted according to this definition.

The reference numbers in the claims are not a part of the claims, but rather used for facilitating the reading thereof These reference numbers should not be interpreted as limiting the claims in any form.

What is claimed is:

1. A disposable feminine hygiene utility (10), comprising:
a sanitary napkin (12) to be placed between an undergarment (26) of a user and a user's body;
a permeable longitudinal sleeve (14), connected to said sanitary napkin (12);
an absorbent pillow (20), enclosed by said permeable longitudinal sleeve (14); and
first and second wires (18), each connected to another end of said absorbent pillow (20), for adjusting a position of said absorbent pillow (20) in relation to said longitudinal sleeve (14), with reference to a feminine anatomic section.

2. A utility according to claim 1, wherein said adjusting is carried out without contact between said absorbent pillow (20) and a user's body, resulting in preventing irritation of said user's body.

3. A utility according to claim 1, wherein said sleeve (14) is structured in said sanitary napkin (12).

4. A utility according to claim 1, wherein said sleeve (14) is sewed into said sanitary napkin (12).

5. A utility according to claim 1, wherein said sleeve (14) is welded in said sanitary napkin (12).

6. A utility according to claim 1, wherein said sleeve (14) is adhered to said sanitary napkin (12).

7. A utility according to claim 1, wherein said absorbent pillow is flattened at the bottom thereof

8. A utility according to claim 1, wherein said absorbent pillow (20) is made of spongy material, thereby allowing said pillow to deform according to a user's body that touches said absorbent pillow (20).

9. A utility according to claim 1, further comprising a pack (16) in which said utility is disposed, thereby keeping said utility sterilized until use.

10. A utility according to claim 9, wherein said pack (16) is designed to allow placing therein a used utility, and keeping thereof sealed.

11. A utility according to claim 1, wherein one side of said absorbent pillow (20) comprises a waxed layer.
